(19) United States
(12) Patent Application Publication
(10) Pub. No.: US 2007/0015922 A1
(43) Pub. Date: Jan. 18, 2007

(54) Process for the preparation of 1,1,3,3-tetraalkylisoindoline starting from n-benzylphthalimide

(75) Inventors: Maria Caldararo, Trecate-Novara (IT); Riccardo Po, Livorno (IT); Marco Ricci, Novaro (IT); Giuliana Schimperna, Novara (IT); Nicoletta Cardi, Meina-Novaro (IT)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: POLIMERI EUROPA S.p.A., Brindisi (IT)

(21) Appl. No.: 10/547,382

(22) PCT Filed: Mar. 1, 2004
(86) PCT No.: PCT/EP04/02071

§ 371 Date: Aug. 29, 2006

(30) Foreign Application Priority Data
Mar. 4, 2003 (IT) MI2003 A 000387

(51) Int. Cl. C07D 209/44; C07F 3/02

(52) U.S. Cl. 548/470; 260/665 G

(57) ABSTRACT

Process for the preparation of 1,1,3,3-tetra-alkyl derivatives of isoindoline which comprises the transformation of N-benzylphthalimide into N-benzyl-1,1,3,3-tetra-alkylisoindoline by means of treatment with a Grignard reagent prepared in methyl-tert-butyl ether starting from magnesium and an alkyl halide and subjecting the intermediate thus obtained to a hydrogenolysis reaction in the presence of hydrogen and a catalyst based on supported palladium.

The present invention relates to a process for the preparation of isoindoline derivatives and, in particular, 1,1,3,3-tetra-alkyl derivatives having general formula (I): 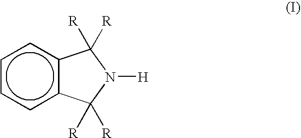
wherein the R groups represent (iso)alkyl radicals containing from 1 to 8 carbon atoms.

Substituted isoindolines can have numerous, interesting applications, for example, as intermediates in the synthesis of pigments or radicalic reaction initiators. For this reason, the synthesis of isoindolines has been the object of vast studies and research.

With respect, in particular, to the synthesis of tetra-alkylisoindolines, these have been prepared, for example, by the cyclodimerization of dipropargylamines and acetylenes in the presence of catalysts based on nickel (G. P. Chiusoli, L. Pallini, G. Terenghi, “Transition Metal Chemistry” 1983, 8, 189) or cobalt (G. P. Chiusoli, L. Pallini, G. Terenghi, “Transition Metal Chemistry” 1984, 9, 360).

Tetra-alkylisoindolines have also been prepared by the carbonylation, in the presence of a catalyst based on palladium, of dipropargylamines. Cyclopentadienone intermediates are formed, which react with ethylene giving adducts which, in turn, can be subsequently decarbonylated and aromatized thus obtaining the desired tetra-alkylisoindolines (G. P. Chiusoli, M. Costa, S. Reverberi, G. Salerno, M. G. Terenghi, “Gazzetta Chimica Italiana” 1987, 117, 695). These synthesis methods however are jeopardized by the necessity of preparing dipropargylamines used as raw material.

Of greater practical interest is the synthesis set up by Rizzardo and collaborators, which comprises the reaction of N-benzylphthalimide with a Grignard reagent as first step. The only example described by Rizzardo consists in the synthesis of 1,1,3,3-tetramethylisoindoline and in this case, the Grignard reagent is prepared starting from methyl iodide and magnesium (6 and 6.25 equivalents, respectively) using, as solvents, first ethyl ether, followed by toluene: after 4 hours at reflux temperature, the intermediate N-benzyl-1,1,3,3-tetramethylisoindoline is obtained with a yield of 37%.

The N-benzyl-1,1,3,3-tetramethylisoindoline is then treated with hydrogen, in glacial acetic acid and in the presence of catalysts based on palladium at 5% on carbon. After 3 hours of reaction at room temperature and under a hydrogen pressure equal to 4 atmospheres, 1,1,3,3-tetramethylisoindoline is obtained (I, R═CH₃) with a yield of 96% (P. G. Griffiths, G. Moad, E. Rizzardo, D. H. Solomon, “Australian Journal of Chemistry” 1983, 36, 397).

Even though, also in this case, N-benzylphthalimide is not a commercial raw material, it can be easily and rapidly prepared, for example by treating the potassium salt of phthalimide with benzyl bromide or chloride or by the reaction of phthalic anhydride with benzylamine. The process of Rizzardo and collaborators, however, has two main drawbacks:
1. due to a renewed attention to safety problems and the new, stricter regulations deriving therefrom, the use of ethyl ether, once widely adopted, currently creates significant problems as a result of its high flammability and the facility with which it gives rise to peroxides which, in turn, create the risk of explosion;
2. although the hydrogen pressure applied in the second passage is low, it obviously requires the use of equipment suitable for operating under pressure, which consequently leads to a more complex processing and, if the equipment is not available, to an increase in costs.

The Applicant has now found that it is possible to prepare 1,1,3,3-tetra-alkylisoindoline according to a process which is substantially simpler than those described so far and which solves the problems relating thereto.

This improved process, described in the enclosed claims, uses, as raw material, N-benzylphthalimide which, as already mentioned, can be easily and rapidly prepared, for example by treating an alkaline salt (for example of potassium) of phthalimide with a benzyl halide, for example, bromide or chloride (yields: 65 and 57%, respectively) or, and preferably, by the reaction of phthalic anhydride with benzylamine (yield: 95%).

The N-benzylphthalimide is thus transformed into an N-benzyl-1,1,1,3,3-tetra-alkylisoindoline by treatment with a Grignard reagent, prepared in methyl-tert-butyl ether starting from magnesium and an alkyl halide.

Alkyl halides which can be used are iodides, and also bromides and chlorides. They are normally used in an equi-molecular quantity with the magnesium or in the presence of a slight excess (up to 10%, but preferably from 3 to 9%) of either of the reagents.

The alkyl halide/N-benzylphthalimide molar ratio, can in turn range from 4 to 10 and can be optimized each time, according to the greater or lesser reactivity of the halide selected and/or of the Grignard reagent generated therefrom. The best results are normally obtained with ratios ranging from 5 to 9.

Particular importance should be given to the selection of the solvent. It is well known, in fact, that Grignard reagents are prepared in ethers. If the reaction with N-benzylphthalimide is carried out in the presence of an ether, however, it is not completed but stops at intermediates products, which contain hydroxyl groups. It is therefore necessary to prepare the Grignard reagent in ether and use it subsequently in another solvent with a higher boiling point (generally an aromatic solvent such as toluene): the ether can then be removed by distillation during the reaction which, in this way, is completed to give the desired products. This requirement makes many of the ethers normally used for preparing Grignard reagents useless, for example butyl ether ([n-C₄H₉]₂O with a boiling point of 142-143° C.) or butyl diglime ([n-C₄H₉OCH₂CH₂]₂O with a boiling point of 256° C.). Although tetrahydrofuran is widely used for the preparation of Grignard reagents, it surprisingly gives low yields in the tetra-alkylation of N-benzylphthalimide. Methyl-tert-butyl ether, on the other hand, is an excellent solvent as, although it is significantly less volatile than ethyl ether, it has an acceptable boiling point (55-56° C.) and does not give rise to the formation of peroxides.

In the last passage of the synthesis, the N-benzyl-1,1,3,3-tetra-alkylisoindolines are treated with hydrogen, in glacial acetic acid and in the presence of catalysts based on palladium at 5% on carbon. It has been surprisingly found that not only is it possible to carry out the reaction at room temperature and atmospheric pressure but that more drastic conditions, and even over-prolonged reaction times, cause a decrease in the yield, probably due to the activation of consecutive reactions. It is therefore possible to carry out the whole synthesis without having to resort to equipment capable of operating under pressure.

The process, object of the present invention, therefore has other advantages such as, for example, the possibility of using, in some cases, only 5 equivalents of Grignard reagent.

The invention is further described by means of the following examples which are provided for purely illustrative purposes and do not limit the scope of the invention itself.

### EXAMPLE 1

### Synthesis of 1,1,3,3-tetramethylisoindoline

### a) Synthesis of N-benzyl-1,1,3,3-tetramethylisoindoline

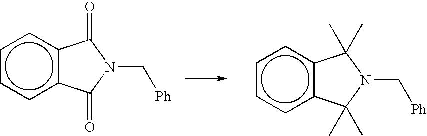

5.9 g (0.24 moles) of magnesium filings and 50 ml of anhydrous methyl-tert-butyl ether are charged, in an inert environment, and 16 ml (36.5 g; 0.26 moles) of methyl iodide diluted in 100 ml of anhydrous methyl-tert-butyl ether is then added dropwise at such a rate as to maintain the solvent at reflux temperature. At the end of the addition, the mixture is heated to maintain the reflux for a further 30 minutes, and a solution of 11.8 g of N-benzylphthalimide (0.05 moles) in 150 ml of anhydrous toluene are then added at such a rate as to maintain a temperature of 80° C. The methyl-tert-butyl ether is contemporaneously removed by distillation. At the end of the addition, the removal of the ether is completed and the mixture is brought to reflux temperature (about 110° C.), stirring it subsequently at this temperature for a further 4 hours. Once the complete conversion of the substrate has been verified (by means of GC and TLC), the reaction mixture is cooled to room temperature, petroleum ether is added, the mixture is stirred in the air for 2 hours (during which the solution becomes purple-coloured) and is then filtered on celite, eluting with petroleum ether. The solvent is removed at reduced pressure from the filtrate (yellow) and the residue obtained is purified using a basic alumina column (activity 1), eluting with petroleum ether/ethyl acetate 99:1.

4.5 g of the intermediate N-benzyl-1,1,3,3-tetramethylisoindoline are obtained with a gas-chromatographic titer equal to 82% (0.014 moles; yield 28%).

### b) Synthesis of 1,1,3,3-tetramethylisoindoline

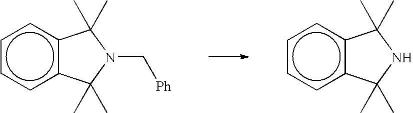

80 ml of glacial acetic acid, 4.5 g of N-benzyl-1,1,3,3-tetramethylisoindoline at 82% (0.014 moles) and 0.8 g of palladium on carbon at 5% are charged, in an inert atmosphere, into a glass reactor. The reactor is placed in a hydrogen atmosphere and the mixture is stirred at atmospheric pressure and room temperature for 3 hours, after which the complete conversion of the substrate is verified by means of GC and TLC. The reaction mixture is filtered on celite and the panel washed with acetic acid. The acetic acid is removed by distilling it at reduced pressure and an oily residue is obtained, to which water is added. It is then washed with ethyl ether and the aqueous solution is basified with an aqueous solution of sodium hydroxide at 10% up to pH 9. The product is extracted with ethyl ether, the extracts are joined, anhydrified on anhydrous sodium sulfate and filtered, the solvent then is removed at reduced pressure thus obtaining 2.5 g of 1,1,3,3-tetramethylisoindoline with a gas-chromatographic titer of 84% (0.012 moles; yield 86%).

### EXAMPLE 2

### Synthesis of 1,1,3,3-tetraethylisoindoline

### a) Synthesis of N-benzyl-1,1,3,3-tetraethylisoindoline

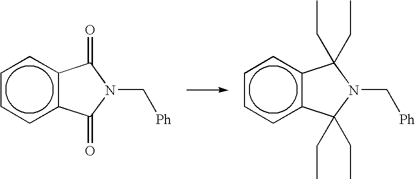

8.75 g (0.36 moles) of magnesium turnings, 20 ml of anhydrous methyl-tert-butyl ether and 2 drops of dibromoethane are charged, in an inert environment. 26.5 ml (38.7 g; 0.36 moles) of ethyl bromide diluted in 100 ml of anhydrous methyl-tert-butyl ether are then added dropwise at such a rate as to maintain the solvent at reflux temperature. At the end of the addition, most of the methyl-tert-butyl ether is evaporated, and a solution of 10 g of N-benzylphthalimide (0.042 moles) in 250 ml of anhydrous toluene is then added at such a rate as to allow a temperature of 80° C. to be reached and maintained. The residual methyl-tert-butyl ether is contemporaneously removed by distillation. At the end of the addition, the removal of the ether is completed and the mixture is brought to reflux temperature (about 110° C.), stirring it subsequently at this temperature for a further 4 hours. Once the complete conversion of the substrate has been verified (by means of GC), the reaction mixture is cooled to room temperature, petroleum ether is added, the mixture is stirred in the air for 2 hours (during which the solution becomes purple-coloured) and is then filtered on celite, eluting with petroleum ether. The filtrate obtained is washed with water until the washings are neutral, it is then anhydrified on sodium sulfate, filtered and dried. A red oil is obtained, which is purified on a basic alumina column (activity 1), eluting with petroleum ether.

5.5 g of the intermediate N-benzyl-1,1,3,3-tetraethylisoindoline are obtained with a gas-chromatographic titer equal to 90% (0.0154 moles; yield 37%).

### b) Synthesis of 1,1,3,3-tetraethylisoindoline

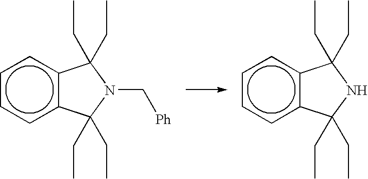

100 ml of glacial acetic acid, 5.5 g of N-benzyl-1,1,3,3-tetraethylisoindoline at 90% (0.0154 moles) and 1 g of palladium on carbon at 5% are charged, in an inert atmosphere, into a glass reactor. The reactor is placed in a hydrogen atmosphere and the mixture is stirred at atmospheric pressure and room temperature for 3 hours, after which the complete conversion of the substrate is verified by means of GC and TLC. The reaction mixture is filtered on celite and the panel washed with acetic acid. The acetic acid is removed by distilling it at reduced pressure and an oily residue is obtained, to which water is added. It is then basified with an aqueous solution of sodium hydroxide at 10% up to pH 9. The product is extracted with ethyl ether, the extracts are joined, anhydrified on anhydrous sodium sulfate and filtered, the solvent then is removed at reduced pressure thus obtaining 3.9 g of 1,1,3,3-tetraethylisoindoline with a gas-chromatographic titer of 88% (0.0148 moles; yield 96%).

1. A process for the preparation of 1,1,3,3-tetra-alkyl derivatives of isoindoline having general formula (I): 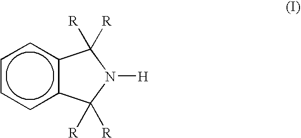
wherein the R groups represent (iso)alkyl radicals containing from 1 to 8 carbon atoms which comprises:
a. preparing the intermediate N-benzyl-1,1,3,3-tetra-alkylisoindoline by treatment in an aromatic solvent of N-benzylphthalimide with a Grignard reagent, prepared in methyl-tert-butyl ether starting from magnesium and an alkyl halide;
b. continuously distilling the methyl-tert-butyl ether solvent during the reaction of step (a);
c. subjecting the intermediate N-benzyl-1,1,3,3-tetraalkyl isoindoline coming from step (b) to hydrogenolysis in the presence of hydrogen and a supported palladium catalyst, at room temperature and atmospheric pressure;
d. recovering the final product having general formula (I).

2. The process according to claim 1, wherein the N-benzylphthalimide is prepared from an alkaline salt of phthalimide with a benzyl halide.

3. The process according to claim 1, wherein the N-benzylphthalimide is prepared by the reaction of phthalic anhydride with benzylamine.

4. The process according to claim 1, 2 or 3, wherein the alkyl halide is used in an excess of up to 10% with respect to the magnesium.

5. The process according to claim 1, 2 or 3, wherein the magnesium is used in an excess of up to 10% with respect to the alkyl halide.

6. The process according to any of the previous claims, wherein the molar ratio alkyl halide/N-benzylphthalimide ranges from 4 to 10.

7. The process according to any of the previous claims, wherein the aromatic solvent of step (a) is toluene.

8. The process according to any of the previous claims, wherein the hydrogenolysis catalyst consists of palladium supported on coal.
